(19) United States
(12) Patent Application Publication
(10) Pub. No.: US 2025/0367086 A1
(43) Pub. Date: Dec. 4, 2025

(54) SULFATE- AND PEG- FREE CLEANSING COMPOSITION WITH HIGH SALICYLIC ACID

(71) Applicant: L'OREAL, Paris (FR)

(72) Inventors: Hungta LIN, Teaneck, NJ (US); Christopher PANG, New York, NY (US); Changlong CHEN, Edison, NJ (US); Mariko HASEBE, New York, NY (US)

(73) Assignee: L'OREAL, Paris (FR)

(21) Appl. No.: 18/680,694

(22) Filed: May 31, 2024

(51) Int. Cl. A61K 8/368; A61K 8/37; A61K 8/44; A61K 8/81; A61Q 19/10

(52) U.S. Cl. CPC A61K 8/368; A61K 8/37; A61K 8/447; A61K 8/817; A61Q 19/10; A61K 2800/33; A61K 2800/596

(57) ABSTRACT

A cleansing composition is for cleansing skin in a rinse-off formulation provides a stable disposition of an acne treatment active, for example, beta hydroxy acid, on the skin that is resistant to removal by rinsing with or without use of a cleansing apparatus. The cleansing composition includes at least one beta hydroxy acid, for example, salicylic acid or a derivative thereof, which is present in an amount that is greater than 1%, by weight, based on the weight of the cleansing composition, and an aqueous carrier that includes lauryl lactate for thickening and stabilization, at least one surfactant, at least one cationic agent, and optional additives. The cleansing composition has a pleasing high foam, is free of drying and irritating sulfates and PEG-type components, demonstrates phase stability up to at least three months at temperatures up to 45° C., and demonstrates retention of beta hydroxy acid active on the skin after cleansing and rinsing.

### FIELD OF THE INVENTION

The present disclosure is directed to a cleansing composition with high salicylic acid content.

### BACKGROUND OF THE INVENTION

Acne is a common skin disorder and is treated using a variety of different agents. Salicylic acid, a mild beta-hydroxy acid, known for facilitating sloughing of dead skin cells and other cellular debris, is often used in treating acne, as well as other skin conditions that include psoriasis, keratoses, and ichthyoses. Commercial forms of cosmetic products that include salicylic acid include peels, cleansers, and leave on treatment in the forms of lotions, creams, and make ups. It is known that salicylic acid exhibits a very low solubility in water, and is prone to re-crystalize, especial at high temperature. Accordingly, a challenge with many of these product forms is that they deliver salicylic acid that is only transiently available on the skin, such as in cleansers and peels, or that is in relatively low amounts, thus precluding sustained effectiveness on the skin for addressing acne and other skin conditions that benefit from the agent.

Compositions with a concentration of salicylic acid that is greater than 1% present the challenge that they are vulnerable to phase separate or crystallization, and are also prone to being hazy with less than 100% transparency. Stable solubilization of salicylic acid in amounts of 1% or more, by weight, can be achieved using solubilizing thickeners and surfactants that contain polyethylene glycol (PEG) and sulfates, which ingredients confer stability of salicylic acid but cause dryness and discomfort for skin, and are increasingly disfavored by consumers. Without sulfates and/or PEG, salicylic acid is vulnerable to precipitation, affecting not only the availability of the active for treatment, but also adversely affecting the stability of the product.

Accordingly, there is a need for a composition that overcomes the shortcomings of the prior art and provides benefits that include a stable formulation with high amounts of salicylic acid that demonstrates the ability to cleanse skin and provide persistent delivery of a skin active, for example, salicylic acid and derivatives thereof. The present invention provides such a composition that surprisingly remains stable in the absence of PEG and sulfates and demonstrates efficacy to deliver and deposit actives to skin, particularly salicylic acid and derivatives thereof.

### BRIEF SUMMARY OF THE INVENTION

In accordance with the various embodiments, a cleansing composition is provided for cleansing the skin in a rinse-off formulation that provides a stable disposition of an acne treatment active, for example, beta hydroxy acid, on the skin that is resistant to removal by rinsing with or without use of a cleansing apparatus. In the various embodiments, the inventive cleansing composition includes at least one beta hydroxy acid, for example, salicylic acid or a derivative thereof, which is present in an amount that is greater than 1%, by weight, based on the weight of the cleansing composition, and an aqueous carrier that includes lauryl lactate for thickening and stabilization, at least one surfactant, at least one cationic agent, and optional additives. The cleansing composition has a pleasing high foam, is free of drying and irritating sulfates and PEG-type components, demonstrates phase stability up to at least three months at temperatures up to 45° C., has a viscosity in a range from about 15 UD to about 50 UD, and demonstrates retention of beta hydroxy acid active on the skin after cleansing and rinsing.

In various embodiments, provided is a cleansing composition that includes:
a) at least one acne treatment active comprising at least one beta hydroxy acid that comprises salicylic acid or a derivative thereof, present in an amount that is greater than 1%, by weight, based on the weight of the cleansing composition; and
b) an aqueous carrier system comprising:
   (i) at least one thickener comprising a lauryl lactate;
   (ii) at least one surfactant;
   (iii) at least one cationic agent; and
   (iv) water,
wherein the cleansing composition is essentially free of PEG-type compounds, PEG-type surfactants, and sulfate-based surfactants.

In some embodiments of the cleansing composition salicylic acid is present in an amount that is in a range from greater than 1% to about 4%, by weight, based on the weight of the cleansing composition.

In some embodiments of the cleansing composition salicylic acid is present in an amount that is in a range from greater than 1% to about 2%, by weight, based on the weight of the cleansing composition.

In some embodiments of the cleansing composition lauryl lactate is present in the cleansing composition from about 0.2% to about 5%, by weight based on the weight of the composition

In some embodiments of the cleansing composition lauryl lactate is present in the cleansing composition at about 0.65%, by weight based on the weight of the composition.

In some embodiments of the cleansing composition the at least one surfactant comprises at least one amphoteric surfactant and at least one anionic surfactant each present in an amount that is in a range from about 0.05% to about 15%, by weight, based on the weight of the cleansing composition.

In some embodiments of the cleansing composition the at least one amphoteric surfactant includes cocamidopropyl hydroxysultaine present in the cleansing composition from about 1% to about 10%, and the at least one anionic surfactant includes sodium methyl cocoyl taurate present in the cleansing composition from about 1% to about 10%, by weight based on the weight of the composition.

In some embodiments of the cleansing composition the at least one amphoteric surfactant includes cocamidopropyl hydroxysultaine present in the cleansing composition at about 7.5%, and the at least one anionic surfactant includes sodium methyl cocoyl taurate present in the cleansing composition at about 4.8%, by weight based on the weight of the composition.

In some embodiments of the cleansing composition the at least one cationic agent is polyquaternium-47 present from about 0.01% to about 5%, by weight based on the weight of the composition.

In some embodiments of the cleansing composition the at least one cationic agent is polyquaternium-47 present at about 0.1%, by weight based on the weight of the composition.

In some embodiments of the cleansing composition the cleansing composition, when applied to a keratinous tissue, demonstrates deposition and retention of the beta hydroxy acid on the keratinous tissue after rinsing with water.

In some embodiments of the cleansing composition the cleansing composition exhibits stability from phase separation and crystallization of the beta hydroxy acid at an ambient temperature of about 45° C. for a period of at least 3 months.

In some embodiments of the cleansing composition the cleansing composition is free of Decyl Glucoside, Peg-150 pentaerythrityl tetrastearate (and) Peg-6 caprylic/capric glycerides, sodium lauryl sulfate, sodium laureth sulfate, sodium lauryl ether sulfate, ammonium lauryl sulfate, ammonium laureth sulfate.

In some embodiments the cleansing composition comprises one or more additives selected from the group consisting of skin care actives, humectants, water-based solvents, pH adjusters, viscosity adjusters, chelating agents, preservatives, cooling agents, fillers, fragrances, dyes, pigments, oils, and combinations thereof.

In some embodiments the cleansing composition comprises one more additives selected from the group consisting of capryloyl salicylic acid, glycerin, sodium hydroxide, zinc gluconate, tetrasodium glutamate diacetate, sodium benzoate, hexylene glycol, sodium chloride, and menthol, and combinations thereof.

In various embodiments, provided is a cleansing composition comprising:
a) at least one acne treatment active comprising at least one beta hydroxy acid comprising salicylic acid present in an amount that is greater than 1%, by weight, based on the weight of the cleansing composition; and
b) an aqueous carrier system comprising:
   (i) at least one thickener comprising a lauryl lactate present in an amount that is in a range from about 0.1% to about 1%;
   (ii) a combination of surfactants comprising at least one amphoteric surfactant and at least one anionic surfactant each present in an amount that is in a range from about 0.05% to about 15%, by weight, based on the weight of the cleansing composition;
   (iii) at least one cationic agent present in an amount that is in a range from about 0.01% to about 0.5%, by weight, based on the weight of the cleansing composition; and
   (iv) water,
wherein the cleansing composition has a viscosity is in a range from about 15 UD to about 50 UD, and is essentially free of PEG-type compounds, PEG-type surfactants, and sulfate-based surfactants.

In some embodiments the cleansing composition comprises the cleansing composition, when applied to a keratinous tissue, demonstrates deposition and retention of the beta hydroxy acid on the keratinous tissue after rinsing with water.

In some embodiments the cleansing composition comprises one more additives selected from the group consisting of capryloyl salicylic acid, glycerin, sodium hydroxide, zinc gluconate, tetrasodium glutamate diacetate, sodium benzoate, hexylene glycol, sodium chloride, and menthol, and combinations thereof.

In some embodiments the cleansing composition exhibits stability from phase separation and crystallization of the beta hydroxy acid at an ambient temperature of about 45° C. for a period of at least 3 months.

In some embodiments the cleansing composition the cleansing composition is free of Decyl Glucoside, Peg-150 pentaerythrityl tetrastearate (and) Peg-6 caprylic/capric glycerides, sodium lauryl sulfate, sodium laureth sulfate, sodium lauryl ether sulfate, ammonium lauryl sulfate, ammonium laureth sulfate.

In various embodiments, provided is a cleansing composition comprising:
a) at least one acne treatment active comprising at least one beta hydroxy acid comprising salicylic acid present in an amount that is from about 1% to about 2%, by weight, based on the weight of the cleansing composition; and
b) an aqueous carrier system comprising:
   (i) at least one thickener comprising a lauryl lactate present at about 0.65%;
   (ii) a combination of surfactants comprising at least one amphoteric surfactant that includes cocamidopropyl hydroxysultaine present in the cleansing composition at about 7.5%, and at least one anionic surfactant that includes sodium methyl cocoyl taurate present in the cleansing composition at about 4.8%, by weight, based on the weight of the cleansing composition;
   (iii) at least one cationic agent that includes polyquaternium-47 present at about 0.1%, by weight based on the weight of the composition;
   (iv) additives comprising capryloyl salicylic acid present at about 0.05%, glycerin present at about 5%, sodium hydroxide present at about 0.4%, zinc gluconate present at about 0.2%, tetrasodium glutamate diacetate present at about 0.05%, sodium benzoate present at about 0.001%, hexylene glycol present at about 0.5%, sodium chloride present at about 2.1%, and menthol present at about 0.1%, all by weight based on the weight of the composition; and
   (v) water;
wherein the cleansing composition has a viscosity is in a range from about 25 UD to about 35 UD, and is essentially free of PEG-type compounds, PEG-type surfactants, and sulfate-based surfactants.

It is to be understood that both the foregoing general description and the following detailed description are exemplary and explanatory only and are not restrictive of the invention.

### DETAILED DESCRIPTION OF THE INVENTION

The cleansing composition overcomes shortcomings in the art pertinent to salicylic acid induced instability in cleansing compositions. While sulfate and PEG based surfactants and solubilizers are required to obtain stability at high amounts of salicylic acid, the inventors have unexpectedly shown that the inventive cleansing composition, as disclosed and as exemplified herein, demonstrates 100% transparency and stability from crystallization, precipitation and phase separation, has pleasing foam, is thick and non-runny, allowing ease of application to skin and retention on skin prior to rinsing, and demonstrates deposition and retention of effective amounts beta hydroxy acid, for example, salicylic acid, even after rinsing.

The benefits of the inventive composition are surprisingly accomplished in the absence of PEG and sulfate containing ingredients that are commonly employed and accepted and understood in the art to be necessary for solubilizing and stabilizing large amounts of SA, which PEG and sulfate containing ingredients are expressly excluded from the inventive composition. The composition has a viscosity in a range from about 15 UD to about 50 UD which is required to confer retention on skin and resistance to runniness prior to rinsing, and more particularly in a range from about 25 UD to about 35 UD. In contrast, exemplified comparative compositions lacking the specific combination of claimed ingredients is unstable, and far more liquidous, demonstrating a negligible viscosity, and thus unsuitable for retention on skin prior to rinsing, and far less effective for deposition of SA on skin. Further, while a Benchmark composition which contains PEG and sulfate ingredients and high amounts of salicylic acid demonstrates stability and viscosity in an acceptable range, it has a viscosity that is less than half of the average viscosity of the embodiments of the inventive compositions, all of which have a viscosity of greater than 30 UD.

The inventors have demonstrated a clear solution to the problems associated with use of less desirable PEG and sulfate containing ingredients for accomplishing solubilization of SA, providing a composition with a pleasant and desirable foamy and smooth feel that avoids the drying effects of PEG and sulfates.

The deposition and enhanced salicylic acid retention was demonstrated herein by visual (unaided eye) observation, deposition was evaluated by in vitro testing on synthetic skin (Bio skin), and foaming was demonstrated by foaming analyzer as discussed herein.

In addition, the inventive cleansing composition demonstrates stability from phase separation, high foaming quality, high transparency (free from hazing) and a mild aesthetic when applied to skin.

### Definitions

The term “skin” as used herein includes skin materials containing keratin such as facial and body skin, scalp, eyebrows, and lips. As referenced in the examples herein, Bio skin is a pseudo skin manufactured substrate that is a substitute for skin and is known in the cosmetics and other industries for its representative similarity to keratinous substrates, such as skin.

“Keratinous substrate” and “keratinous tissue” each includes but is not limited to skin, hair, and nails.

The term “cleanser” as used herein can be any cleansing composition utilized for application to a keratinous tissue for cleansing the skin, removal of make-up and the like.

“Natural” or “Nature-based” as used herein means and refers to cosmetically acceptable materials and components that are one or more of directly obtained from nature, are obtained from nature with minimal processing, and are derivatives of materials that are obtained from nature. The cleansing composition according to the instant disclosure is, in some embodiments, up to 99.11% natural, or “Highly natural” or “All Natural” which means that all carbon atoms of intentionally added ingredients are from natural sources per ISO 16128.

References to “foaming” and “high foaming” as used herein means visually observed foaming and measurement of the absolute or relative height of a formulation after agitation and measurement of generated foam height using an analyzer (for example Kruss DFA100 Dynamic Foam Analyzer).

As used herein with respect to the foaming property of cleansing composition, including the cleansing composition, the term “foaming” means and refers to the capacity of a composition (inventive or comparative) to develop a foam upon rubbing that would be associated with applying and gently scrubbing skin or other keratinous substrates with the formulation. Reference is made to a known testing method for establishing foam formation in vitro employing equipment that is well known in the art, namely analysis using a Krauss DFA100™ Dynamic Foam Analyzer, which provides a relative measure of achieved foam formation, as more specifically described herein. In some examples herein, foaming is described as relative formation of foam density per unit area when compared to other tested formulations under the same conditions. In some examples, bubble count using the indicated foam analyzer is provided. Foaming Capacity, or good foaming or high foaming as used herein relative to foaming on a keratinous substrate, such as facial skin, is based on a scale from low to high.

“Stable” and “Stability” as used herein means that the cleansing composition does not show any signs of significant changes in one or more of color, odor, pH, or viscosity at room temperature and at temperatures in the range from about 25° C. to about 45° C. In various embodiments, an inventive composition remains stable at temperatures in the range from about 25° C. to about 45° C., over a time period of at least one month, or at least two months, and up to at least three months, or any value, range, or sub-range therebetween. The cleansing composition is stable at 45° C. for three months, wherein stability includes clear appearance (100% transparent) with no hazy appearance, no loss of viscosity, and no crystal formation or precipitation, for example no precipitation of salicylic acid. For example, as described herein, use of certain nature based thickeners, such as chitosan, polyethylenimine (PEI), or polylysine, resulted in unstable compositions in which there was a distinct phase separation.

References to “free from hazing” as used herein means that the cleansing composition are essentially 100% clear, likely representing that essentially all of the salicylic acid is maintained as solubilized, clarity, or free from haze, which may be confirmed visually based on the absence of crystals and the absence of haze, and more precisely using a spectrophotometer or similar instrument to measure the type and amount of the light that passes through wherein 100% of Light Transmission (using, for example, a Varian Cary 5000 uv-vis-nir spectrophotometer) is equivalent to =100% clear and free from haze.

The cleansing composition, can comprise, consist of, or consist essentially of the essential elements and limitations of the invention described herein, as well as any additional or optional ingredients, components, or limitations described herein or otherwise useful.

### Cleansing Composition

In accordance with the various embodiments, the cleansing composition is suitable for application to keratinous tissue for cleansing, particularly for addressing acne. The cleansing composition is free of, or substantially free of, or devoid of one or more of sulfates, including but not limited to sulfate-based or sulfate-containing surfactants, and PEG-type compounds, including PEG-type thickeners and surfactants. Other materials may be included or excluded as described herein below under “excluded materials.”

### Beta Hydroxy Acid

In accordance with the various embodiments, the cleansing composition comprises at least one acne treatment active comprising beta hydroxy acid. In some embodiments, the cleansing composition includes a beta hydroxy acid comprising salicylic acid.

In some embodiments according to the disclosure, the cleansing composition includes salicylic acid present in the cleansing composition in an amount that ranges from about 0.2% to about 2% by weight, based on the total weight of the cleansing composition.

In some embodiments, the beta hydroxy acid is salicylic acid present in the cleansing composition at about 1%, or about 1.5%, or about 2%, by weight based on the weight of the composition.

The term “beta-hydroxy acid” is understood to mean, according to the present invention, a carboxylic acid having a hydroxyl functional group and a carboxylic functional group separated by two carbon atoms. A beta hydroxy acid can be present in the cleansing composition in the form of the free acid and/or in the form of one of its associated salts (salts with an organic base or an alkali metal, in particular), especially according to the final pH imposed on the cleansing composition.

The beta hydroxy acid is selected from one or more of salicylic acid and derivatives thereof (including 5-n-octanoylsalicylic acid, salicylate, sodium salicylate, and willow extract), capryloyl salicylic acid, beta hydroxybutanoic acid, tropic acid, and trethocanic acid.

The total amount of beta hydroxy acid includes, in some embodiments, from about 0.1% up to and not more than about 4%, or not more than about 3%, or not more than about 2% of beta hydroxy acid, by weight, based on the total weight of the cleansing composition, including increments and all ranges and subranges therein and there between, by weight, based on the total weight of the cleansing composition. One of ordinary skill in the art, however, will appreciate that other ranges are within the scope of the invention.

In some embodiments, the cleansing composition includes and up to and not more than about 2%, or about 1.9% of beta hydroxy acid. In some embodiments, the cleansing composition includes from about 0.1% to about 1% of beta hydroxy acid, or from 1% up to about 1.5%. In some embodiments, the cleansing composition includes and up to and not more than about 1% of beta hydroxy acid, or not more than about 1.5%. In some embodiments, the cleansing composition includes more than about 2% beta hydroxy acid.

Thus, the at least one beta hydroxy acid is present in the cleansing composition from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 weight percent, including increments and all ranges and subranges therein and there between.

### Thickener

In accordance with the various embodiments, the cleansing composition comprises at least one thickener comprising a lauryl lactate.

In various embodiments, lauryl lactate is demonstrated to provide the benefits that are at least equivalent to a PEG-type thickener while satisfying the demands of consumers seeking more natural products with sustainable ingredients. Accordingly, in various embodiments, cleansing composition expressly excludes PEG-type thickeners, including, but not limited to, PEG-7 glyceryl cocoate, PEG-60 hydrogenated castor oil, and PEG-150 pentaerythrityl tetrastearate (and) PEG-6.

In some embodiments, lauryl lactate is present in the cleansing composition at about 0.65%, by weight based on the weight of the composition.

In various embodiments lauryl lactate is present in an amount in the range of from about 0.2% to about 5%. In some embodiments, lauryl lactate is present in an amount that is at least 0.2%, or about 0.5%, or about 0.75%, or about 1%, up to about 5%, or about 0.1% to about 1%, or to about 0.2% or to about 0.8%, or about 0.5% to about 0.7%, or any value, range, or sub-range therebetween by weight, based on the weight of the cleansing composition.

Thus, in various embodiments, lauryl lactate is present in a composition according to the disclosure from 0.2, 0.3, 0.4, 0.5, 0.6, 0.7, 0.8, 0.9, 1, 2, 3, 4, to about 5 percent, including increments and ranges therein and there between.

### Cationic Agent

In some embodiments, the cleansing composition may include a cationic agent, for example a natural or synthetic cationic polymer.

In some embodiments, the cationic agent is selected from polyquaterniums. In some embodiments, the cationic agent is a polyquaternium comprising or consisting of polyquaternium-47. In some embodiments, polyquaternium-47 is present in the cleansing composition at about 0.1%, by weight based on the weight of the composition.

In some embodiments, the cleansing composition is free or essentially free from any cationic agent. In some embodiments, the cleansing composition is free or essentially free from any cationic agent that is not a polyquaternium. In some specific examples, the cleansing composition is free from cationic agents selected from the group consisting of chitosan, PEI, polylysine, and combinations thereof.

In some embodiments, the cationic agent may be selected from nature-based polymers or may be selected from synthetic polymers. In some examples, the synthetic polymers are non-silicone based.

More generally, cationic agents may be selected from nature-based polymers that are polysaccharides, other natural (i.e., plant, animal, or bacterial based), synthetic, or modified cationic nature-based or synthetic polymers. In some embodiments, the cleansing composition is free or essentially free of cationic polymers selected from chitosan, chitosan derivatives, chitin, starch, starch derivatives, cellulose (ethylcellulose, nitrocellulose, hemicellulose, and hemicellulose derivatives), alginates, mannans, xylans, lignins, arabans, galacturonans, alginate-based compounds, chitin, glucuronoxylans, arabinoxylans, xyloglucans, glucomannans, fructosans such as inulin, pectic acids and pectins, arabinogalactans, agars, glycosaminoglycans, gum arabics, tragacanth gums, ghatti gums, karaya gums, locust bean gums, xanthan gums, mucopolysaccharides, chondroitin sulfates, cationic acrylic polymers, polyacrylates, for example, polyacrylate-1, polyacrylate-2, polyacrylate-3, polyacrylate-4, polyacrylate-16, polyacrylate-17, polyacrylate-18, polyacrylate-19, polyacrylate-21, and any combination of these.

In some embodiments the cationic agent may be selected from poly amines such as polyethylenimine and polylysine.

In some embodiments the cationic agent may be selected from of polyquaterniums, that is polymers having quaternary ammonium centers in the polymer, for example, including polyquaternium-47 (1-Propanaminium, N,N,N-trimethyl-3-[(2-methyl-1-oxo-2-propenyl)amino]-, chloride, polymer with methyl 2-propenoate and 2-propenoic acid). Other non-limiting examples of such polyquaternium compounds may be selected from diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquaternium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acrylamide/dimethyldiallylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name: Polyquaternium-53); Polyquaternium-30; Polyquaternium-35; Polyquaternium-45; Polyquaternium-47; Polyquaternium-50; Polyquaternium-54; Polyquaternium-57; Polyquaternium-63; Polyquaternium-74; Polyquaternium-76; Polyquaternium-86; Polyquaternium-89; Polyquaternium-95; Polyquaternium-98; Polyquaternium-104; Polyquaternium-111; Polyquaternium-112, and mixtures thereof. In some embodiments, the cationic polymer is a synthetic polymer selected from cationic acrylic polymers, for example, an amorphous functional acrylic polymer grafted onto a polyethylene backbone such as SYNTRAN™ 5330, which is a quaternary modified olefin grafted technology.

In some embodiments, the cationic agent may be selected from cationic polymers comprising polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof. Such (co) polymers, or similar (co) polymers, can be combined individually or with other (co) polymers in such a way to form suitable bimodal agents having both cationic and anionic functionalities.

In the various embodiments, the amount of each of the at least one cationic agent that may be present in the cleansing composition can range from about 0.01% to about 10%, or from about 0.01% to about 5%, or from about 0.01% to about 0.5%, or from about 0.1% to about 0.5%, or from about 0.1% to about 0.2%, or from about 0.1% to about 5% or from about 1% to about 3.5%, or from about 0.5% to about 2%, or about 1%, or any suitable combination, sub-combination, range, or sub-range thereof by weight, based on the weight of the cleansing composition. One of ordinary skill in the art, however, will appreciate that other ranges are within the scope of the invention.

Thus, any one of the at least one cationic agent, when present, is present, by weight, based on the total weight of the cleansing composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, to about 10 weight percent, including increments and ranges therein and there between.

### Surfactants

In accordance with the various embodiments, the cleansing composition comprises at least one surfactant. In some embodiments, the cleansing composition comprises more than one surfactant. The at least one surfactant may be an nonionic, cationic, anionic, or an amphoteric or zwitterionic surfactant. In some embodiments, the composition includes one or a combination of surfactants selected from the group consisting of amphoteric, nonionic, anionic surfactants and combinations thereof.

In some embodiments, the cleansing composition comprises a combination of surfactants including at least one amphoteric surfactant and at least on anionic surfactant. The at least one amphoteric surfactant may selected from the group consisting of sultaines, betaines, amphoacetates, amphoproprionates, sarcosinates, and combinations thereof, and at least one anionic surfactant may selected from non-sulfate anionic surfactants, such as, for example, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids. In some embodiments, the cleansing composition comprises more than one surfactant of each amphoteric and anionic surfactants.

In some embodiments, the composition comprises at least one surfactant comprising one or more of coco-betaine, cocamidopropyl hydroxysultaine and sodium methyl cocoyl taurate.

In some embodiments, the cleansing composition comprises a combination of surfactants, wherein when coco-betaine is present, at least one of cocamidopropyl hydroxysultaine or sodium methyl cocoyl taurate is present.

In some embodiments, the cleansing composition comprises a combination of surfactants comprising coco-betaine with one or both of cocamidopropyl hydroxysultaine and sodium methyl cocoyl taurate.

In some embodiments, the cleansing composition comprises a combination of surfactants comprising cocamidopropyl hydroxysultaine and sodium methyl cocoyl taurate. In some such embodiments, cocamidopropyl hydroxysultaine is present in the cleansing composition at about 7.5%, and sodium methyl cocoyl taurate is present in the cleansing composition at about 4.8%, by weight based on the weight of the composition.

In various embodiments, each amphoteric surfactant, when present, may be present in an amount in the range of from about 0.05% to about 15%. In some embodiments, each of the at least one amphoteric surfactant is present in an amount from about 1% to about 10%, or from about 5% to about 8%, or about 8%, or any value, range, or sub-range therebetween by weight, based on the weight of the cosmetic cleansing composition.

In various embodiments, each anionic surfactant, when present, may be present in an amount in the range of from about 0.05% to about 15%. In some embodiments, each of the at least one anionic surfactant is present in an amount from about 0.5% to about 10%, or from about 2% to about 8%, or about 5%, or any value, range, or sub-range therebetween by weight, based on the weight of the cosmetic cleansing composition.

Thus, in various embodiments, each surfactant may be present in a composition according to the disclosure from 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, to about 15 percent, including increments and ranges therein and there between. And In various embodiments, the total amount of surfactant including, in some embodiments, at least one amphoteric, at least one anionic surfactant, and combinations of surfactants, may be present in a range from about 0.05% to about 30% or more.

### Amphoteric Surfactant

In the various embodiments, the cleansing composition may comprise at least one amphoteric surfactant (or zwitterionic surfactant). In some embodiments, the cleansing composition comprises more than one amphoteric surfactant. In some embodiments, the cleansing composition may include or excludes any one or more of nonionic, cationic, or anionic surfactants, and in embodiments, excludes PEG/POE surfactants. In some embodiments, the cleansing composition includes a combination amphoteric surfactants.

In some embodiments, the at least one amphoteric surfactant may include cocamidopropyl hydroxysultaine, cocamidopropyl betaine, coco betaine, sodium lauroamphoacetate, disodium cocoamphodiacetate, and combinations thereof.

In some embodiments the at least one amphoteric surfactant may be selected from, for example, betaines, alkyl sultaines, alkyl amphoacetates and alkyl amphodiacetates, alkyl amphoproprionates, amphocarboxylates, alkyl betaines, amidoalkyl betaines, amphophosphates, phosphobetaines, pyrophosphobetaines, carboxyalkyl polyamines, amidoalkyl sultaines, salts thereof, and combinations thereof.

Betaines which can be used in the current compositions include those having the formulas below:

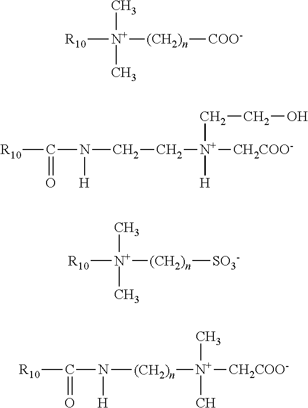

wherein
R¹⁰ is an alkyl group having 8-18 carbon atoms; and
n is an integer from 1 to 3.

Particularly useful betaines include, for example, coco betaine, cocoamidopropyl betaine, lauryl betaine, laurylhydroxy sulfobetaine, lauryldimethyl betaine, cocoamidopropyl hydroxysultaine, behenyl betaine, capryl/capramidopropyl betaine, lauryl hydroxysultaine, stearyl betaine, and mixtures thereof. Typically, the at least one betaine compound is selected from the group consisting of coco betaine, cocoamidopropyl betaine, behenyl betaine, capryl/capramidopropyl betaine, lauryl betaine, and mixtures thereof, and more typically coco betaine.

Hydroxyl sultaines useful in the compositions of the invention include the following

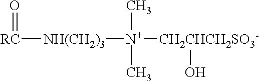

wherein
R is an alkyl group having 8-18 carbon atoms.

More specific examples include, but are not limited to cocamidopropyl hydroxysultaine, lauryl hydroxysultaine, and combinations thereof.

Useful alkylamphoacetates include those having the formula

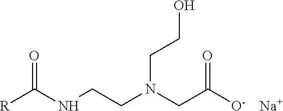

wherein
R is an alkyl group having 8-18 carbon atoms.
useful alkyl amphodiacetates include those having the formula

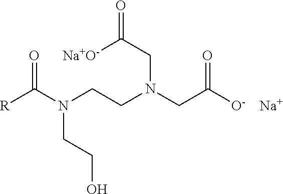

wherein
R is an alkyl group having 8-18 carbon atoms.

### Alkyl Amphopropionates

Exemplary and non-limiting examples of useful alkyl amphopropionates include cocoamphopropionate, caprylamphopropionate, cornamphopropionate, caproampho-propionate, oleoamphopropionate, isostearoamphopropionate, stearoamphopropionate, lauroamphopropionate, salts thereof, and combinations thereof.

The at least one amphoteric surfactant of the present disclosure may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

### Anionic Surfactants

As used herein, the term “anionic surfactant” means a surfactant comprising, as ionic or ionizable groups, only anionic groups. These anionic groups are preferably chosen from the groups CO₂H, CO₂⁻, SO₃H, SO₃⁻, OSO₃H, OSO₃⁻O₂PO₂H, O₂PO₂H, and O₂PO₂²⁻.

Anionic surfactants chosen from non-sulfate anionic surfactants, such as, for example, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

Useful alkyl sulfonates include those of formula (VI):

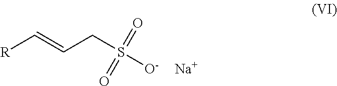

wherein R is selected from H or alkyl chain that has from 1 to 30 carbon atoms, such as from 6 to 24 carbon atoms, for example from 8 to 20 carbon atoms, said chain being saturated or unsaturated, linear or branched. It should be noted that although sodium is shown as the cation in the above formula (VI), the cation may be any alkali metal ion such as sodium or potassium, ammonium ions, or alkanolammonium ions such as monoethanolammonium or triethanolammonium ions. In some instances, the alkyl sulfonate(s) are selected from C8-C16 alkyl benzene sulfonates, C10-C20 paraffin sulfonates, C10-C24 olefin sulfonates, salts thereof, and combinations thereof.

By way of non-limiting example, alkyl sulfonates may be chosen from alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

Non-limiting examples of useful alkyl sulfosuccinates include those of formula (VII):

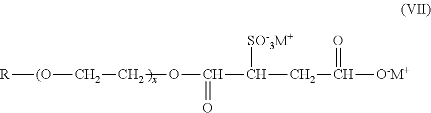

wherein R is a straight or branched chain alkyl or alkenyl group having from 10 to 22 carbon atoms, such as 10 to 20 carbon atoms, and M⁺ is a cation which can independently of each other be, for example, any alkali metal ion such as sodium, potassium, or ammonium, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof.

Exemplary and non-limiting alkyl sulfoacetates include C4-C18 fatty alcohol sulfoacetates and/or salts thereof, such as sodium lauryl sulfoacetate. Useful cations for the salts include any alkali metal ion such as sodium or potassium, ammonium ions, or alkanolammonium ions such as monoethanolammonium or triethanolammonium ions.

Non-limiting examples of alkoxylated monoacids include compounds corresponding to formula (VIII): R—O[CH₂O]ᵤ[(CH₂)ₓCH(R′)(CH₂)_{y}(CH2)_{z}O]ᵥ[CH₂CH₂O]_{w}CH₂—COOH (VIII)
wherein:
R is a hydrocarbon radical containing from about 6 to about 40 carbon atoms;
u, v, and w, independently of one another, represent numbers of from 0 to 60;
x, y, and z, independently of one another, represent numbers of from 0 to 13;
R′ represents hydrogen or alkyl; and
the sum of x+y+z>0.

Compounds corresponding to formula (VIII) may be obtained by alkoxylation of alcohols R—OH with ethylene oxide as the sole alkoxide, or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (VIII), R is linear or branched, acyclic or cyclic, saturated or unsaturated, aliphatic or aromatic, substituted or unsubstituted. R may be a linear or branched, acyclic C6-40 alkyl or alkenyl group, or a C1-40 alkyl phenyl group, for example a C8-22 alkyl or alkenyl group or a C4-18 alkyl phenyl group, such as a C12-18 alkyl group or alkenyl group or a C6-16 alkyl phenyl group; u, v, w, independently of one another, may be a number from 2 to 20, for example a number from 3 to 17, such as a number from 5 to 15; x, y, z, independently of one another, may be a number from 2 to 13, for example a number from 1 to 10, such as a number from 0 to 8.

By way of example only, useful alkoxylated monoacids include Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and combinations thereof. In some cases, preferred ethoxylated acids include Oleth-10 Carboxylic Acid, Laureth-5 Carboxylic Acid, Laureth-11 Carboxylic Acid, and combinations thereof.

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of useful acyl amino acids include those of formula (IX):

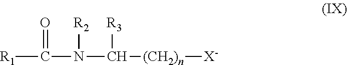

wherein R, R1, R2, and R3 are each independently selected from H or an alkyl chain having from 1 to 30 carbon atoms, said chain being saturated or unsaturated, linear or branched, and X is COO⁻ or SO₃⁻.

By way of example, useful acyl amino acids include acyl taurates, acyl glycinates, acyl glutamates, acyl sarcosinates, salts thereof, and combinations thereof.

Exemplary useful acyl taurates include those of formula (X):

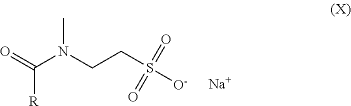

wherein R is selected from H or an alkyl chain having from 1 to 30 carbon atoms, such as from 6 to 24 carbon atoms, for example from 8 to 20 carbon atoms, said chain being saturated or unsaturated, linear or branched. It should be noted that although sodium is shown as the cation in the above formula (X), the cation may be any alkali metal ion such as sodium or potassium, ammonium ions, or alkanolammonium ions such as monoethanolammonium or triethanolammonium ions. Non-limiting examples of acyl taurate salts include sodium cocoyl taurate, sodium methyl cocoyl taurate, and combinations thereof.

Exemplary useful acyl glycinates include those of formula (XI):

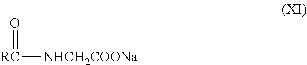

wherein R is an alkyl chain of 8 to 16 carbon atoms. It should be noted that although sodium is shown as the cation in the above formula (XI), the cation may be any alkali metal ion such as sodium, potassium, or ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and combinations thereof.

Exemplary useful acyl glutamates include those of formula (XII):

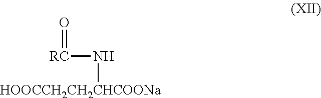

wherein R is an alkyl chain of 8 to 16 carbon atoms. It should be noted that although sodium is shown as the cation in the above formula (XII), the cation may be any alkali metal ion such as sodium, potassium, or ammonium ions, or alkanolammonium ions such as monoethanolammonium or triethanolammonium ions. Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, disodium cocoyl glutamate, and combinations thereof.

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations thereof.

When the anionic surfactant(s) are in salt form, they may be chosen especially from alkali metal salts such as the sodium or potassium salt and preferably the sodium salt, ammonium salts, amine salts and in particular amino alcohol salts, or alkaline-earth metal salts such as the magnesium salt. Examples of amino alcohol salts that may especially be mentioned include monoethanolamine, diethanolamine and triethanolamine salts, monoisopropanolamine, diisopropanolamine or triisopropanol-amine salts, 2-amino-2-methyl-1-propanol salts, 2-amino-2-methyl-1,3-propanediol salts and tris(hydroxymethyl)aminomethane salts. Alkali metal or alkaline-earth metal salts and in particular the sodium or magnesium salts may be used. Exemplary salts of C₆-C₂₄ alkyl monoesters of polyglycoside-polycarboxylic acids include C₆-C₂₄ alkyl polyglycoside-citrates, C₆-C₂₄ alkyl polyglycoside-tartrates and C₆-C₂₄ alkyl polyglycoside-sulfo succinates.

### Water

In accordance with the various embodiments, the cleansing composition includes water.

In various embodiments, the cleansing composition comprises from about 5% to about 90% water, or from about 20% to about 85% water, or from about 50% to about 85% water, or from about 75% to about 80%, water, including increments and all ranges and subranges therein and there between, by weight, based on the total weight of the cleansing composition. One of ordinary skill in the art, however, will appreciate that other ranges are within the scope of the invention. Thus, water is present, by weight, based on the total weight of the cleansing composition, from about 5, 10, 15, 20, 25, 30, 35, 40, 45, 50, 55, 60, 65, 66, 67, 68, 69, 70, 71, 72, 73, 74, 75, 80, 85, to about 90 weight percent, including increments and all ranges and subranges therein and there between.

The water used in the cleansing composition may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

### Excluded Materials

It will be appreciated that in some embodiments, the cleansing composition may include may be substantially or essentially free of or may exclude certain ingredients, for example, sulfates, sulfate surfactants, PEG-type components, PEG-type thickeners and surfactants, petrochemical based compounds, silicone compounds, cationic agents, cationic agents that include chitosan or polyamines including PEI and polylysine, cationic agents that are not polyquaternium based, and combinations thereof. In some embodiments, the cleansing composition is essentially free of or devoid of any one or more of these components.

As used herein, the terms “substantially free” or “essentially free” as used herein mean the specific material may be present in small amounts that do not materially affect the basic and novel characteristics of the embodiments of the cleansing composition according to the disclosure or the material may be absent. For instance, there may be less than 2% by weight of a specific material added to a composition, based on the total weight of the compositions (provided that an amount of less than 2% by weight does not materially affect the basic and novel characteristics of embodiments of the cleansing composition according to the disclosure. Similarly, the compositions may include less than 2%, less than 1.5%, less than 1%, less than 0.5%, less than 0.1%, less than 0.05%, or less than 0.01%, or none (0%) of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition

“PEG-type components,” including, but not limited to, “PEG-type polymeric thickeners” means and refers to excluded components, including thickeners, that have not been added as a component in the cleansing composition. In some embodiments, the cleansing composition is essentially free, free or devoid of one or more of PEG/PEO polymeric thickeners. In some embodiments, the cleansing composition is essentially free of or excludes PEG-type polymeric thickeners selected from PEG-150 pentaerythrityl tetrastearate, PEG-120 thickeners, PEG-150 thickeners, PEG-80 glyceryl tallow ester, PEG-8 PPG (polypropylene glycol)-3 diisostearate, PEG-200 hydrogenated glyceryl palmitate, PEG-n (n=6, 8, 12) beeswax, PEG-4 isostearate, PEG-n (n=3, 4, 8, 150) distearate PEG-n (n=28, 200) glyceryl tallow ester, PEG-7 hydrogenated castor oil, PEG-40 jojoba oil, PEG-120 methyl glucose dioleate, PEG-150 pentaerythritol stearate, PEG-55 propylene glycol oleate, PEG-160 sorbitan triisostearate, SMDI copolymer (polyethylene glycol-150/decyl/methacrylate copolymer), PEG-150/stearyl/SMDI copolymer, PEG-90, and combinations thereof.

In some embodiments, cleansing composition may also exclude one or more PEG type compound selected from poloxamer 184 (polyoxyethylene polyoxypropylene glycol), PEG-20 glyceryl triisostearate, PEG-7 glyceryl cocoate, PEG-20 methylglucoside sesquistearate, PG-5 dioleate, PG-4 diisostearate, PG-10 isostearate, PEG-60 hydrogenated castor oil, PEG-8 stearate, PEG-8 isostearate, PEG-60 hydrogenated castor oil, PEG-150 Laurate, PEG-150 Distearate, PEG-150 pentaerythrityl tetrastearate, PEG-78 Glyceryl Cocoate, PEG-30 Glyceryl Cocoate, PEG-type polymeric thickeners selected from PEG-150 pentaerythrityl tetrastearate, PEG-120 thickeners, PEG-150 thickeners, PEG-80 glyceryl tallow ester, PEG-8 PPG (polypropylene glycol)-3 diisostearate, PEG-200 hydrogenated glyceryl palmitate, PEG-n (n=6, 8, 12) beeswax, PEG-4 isostearate, PEG-n (n=3, 4, 8, 150) distearate PEG-n (n=28, 200) glyceryl tallow ester, PEG-7 hydrogenated castor oil, PEG-40 jojoba oil, PEG-120 methyl glucose dioleate, PEG-150 pentaerythritol stearate, PEG-55 propylene glycol oleate, PEG-160 sorbitan triisostearate, SMDI copolymer (polyethylene glycol-150/decyl/methacrylate copolymer), PEG-150/stearyl/SMDI copolymer, PEG-90, -20, Ceteth-10, Ceteth-20, Isoceteth-20, Laureth-4, Laureth-23, Oleth-10, Oleth-20, Steareth-10, Steareth-20, Steareth-100, and Steareth-21, PEG-20 glyceryl laurate, PEG-40 hydrogenated castor oil, or combinations thereof.

In some embodiments, the cleansing composition is free or essentially free of sulfate-based surfactants. If present, sulfate-based anionic surfactants may be chosen from, for example, alkyl sulfates, alkyl ether sulfates, alkylamido ether sulfates, alkylaryl polyether sulfates, monoglyceride sulfates, or combinations thereof. In various embodiments, the anionic surfactant may be chosen from (C₆-C₂₄)alkyl sulfates, (C₆-C₂₄)alkyl ether sulfates, which are optionally ethoxylated, comprising from 2 to 50 ethylene oxide units, and combinations thereof, in particular in the form of alkali metal salts or alkaline-earth metal salts, ammonium salts or amino alcohol salts. More preferentially, the anionic surfactant(s) are chosen from (C₁₀-C₂₀)alkyl ether sulfates, and in particular sodium lauryl ether sulfate, optionally containing 2.2 mol of ethylene oxide. In other embodiments, sodium lauryl sulfate, sodium lauryl ether sulfate, ammonium lauryl sulfate, ammonium lauryl ether sulfate, sodium laureth sulfate, or combinations thereof may be chosen.

The terms “essentially free of sulfate,” “essentially free of sulfate-based surfactant,” “essentially free of sulfate-based surfactant,” and “essentially sulfate-free” refer to the essential absence of sulfate contents in the cleansing composition of the present invention, and that sulfates and sulfate surfactants have not been added to the cleansing composition as a component. In reference to some embodiments, the term “sulfate-free” means that sulfate has not been added as a component. In reference to some embodiments, the cleansing composition is devoid of sulfate. In reference to some embodiments, the cleansing composition is free or devoid of sulfates that may include, but are not limited to, anionic alkyl sulfates and alkyl ether sulfates. In reference to some embodiments, while it is preferred that no sulfate-based surfactants are present in the cleansing composition, it is possible to have very small amounts of sulfate-based surfactants in the cleansing composition of the invention, provided that these amounts do not materially affect the advantageous properties of the cleansing composition, in particular with respect to mildness to skin. Thus, a sulfate-based surfactant can be present in the cleansing composition at an amount of less than about 0.2% by weight, or less than about 0.1% by weight, or less than about 0.05% by weight, or about 0% by weight, based on the total weight of the cleansing composition.

“Petrochemical-free” means that excluded petrochemical have not been added to the cleansing composition as a component. Some specific but non-limiting examples of petrochemicals that are lacking from the cleansing composition include benzalkonium chloride, isododecane, isohexadecane and the like. In some embodiments, the cleansing composition is not free from petrochemicals, but is formulated to have an amount of petrochemicals that is at or below the amounts found in similar compositions that are commercially available.

“Silicone-free” means that excluded silicones have not been added to the cleansing composition as a component. In some embodiments, a composition is devoid of silicones. Some specific but non-limiting examples of silicones that are lacking from the cleansing composition includes, but is not limited to, silicone polymers, for example selected from dimethicone, cyclopentasiloxane and other silicone oils, and silicone elastomers.

### Optional Additives

In some embodiments, the cleansing composition further comprises one or more additives selected from the group consisting of skin care actives, humectants, water-based solvents, pH adjusters, viscosity adjusters, chelating agents, preservatives, cooling agents, fillers, fragrances, dyes, pigments, oils, and combinations thereof.

In some embodiments, the cleansing composition includes one more additives selected from the group consisting of capryloyl salicylic acid, glycerin, sodium hydroxide, zinc gluconate, tetrasodium glutamate diacetate, sodium benzoate, hexylene glycol, sodium chloride, and menthol.

In some embodiments, the cleansing composition includes each of the additives comprising capryloyl salicylic acid present at about 0.05%, glycerin present at about 5%, sodium hydroxide present at about 0.4%, zinc gluconate present at about 0.2%, tetrasodium glutamate diacetate present at about 0.05%, sodium benzoate present at about 0.001%, hexylene glycol present at about 0.5%, sodium chloride present at about 2.1%, and menthol present at about 0.1%, all by weight based on the weight of the composition.

In some embodiments, the cleansing composition may include at least one additive used in the cosmetics field which does not affect the properties of the cosmetic cleansing composition according to the invention, such as, fragrances, pH adjusters (citric acid, sodium chloride, lactic acid); neutralizing or pH-adjusting agents (e.g., triethylamine (TEA) and sodium hydroxide, Trisodium ethylenediamine disuccinate, and combinations thereof), other cosmetically acceptable additives, such as but not limited to, pearlescent agents, silica, and coloring materials; essential oils; fruit extracts, for example, Pyrus Malus (Apple) Fruit Extract, and Aloe Barbadensis Leaf Juice Powder; actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants (phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; conditioning agents such as C12-15 Alkyl lactate and C12-15 Alcohols; Thickeners, for example Ammonium polyacryloyldimethyl taurate, Propylene glycol and PEG-55 propylene glycol oleate; viscosity adjusters, for example, Sodium chloride and Hexylene Glycol; cooling agents such as menthol; fillers, for example Kaolin; or any combination thereof. Although the optional additives are given as examples, it will be appreciated that other optional components compatible with cosmetic applications known in the art may be used.

In the various embodiments, the amount of one or more additives, alone or in combination, present in the cosmetic cleansing composition can be present in a range from about 0.0001% to about 20%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the cosmetic cleansing composition. One of ordinary skill in the art, however, will appreciate that other ranges are within the scope of the invention.

More generally, it will be appreciated by a skilled artisan that any preservatives, humectants, solvents, oils, or other additives are present only to the extent and in amounts that do not materially adversely affect the basic and novel characteristic(s) of the claimed invention.

In some embodiments, the cleansing composition may include one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the cleansing compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

In some embodiments, the cosmetic cleansing composition may include one or more oils. In some embodiments, the cosmetic cleansing composition is essentially free, free of or devoid of oils. In some examples, the oils may be chosen from hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the cosmetic cleansing composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, musk rose oil, and essential oils, such as Helianthus annuus Seed Oil, Lavandula angustifolia (lavender) Oil, Mentha piperita Oil, Rosmarinus officinalis (rosemary) Leaf Oil Pelargonium graveolens flower oil, Citrus aurantium dulcis (orange) peel oil, Menthe viridis (spearmint) leaf oil, Citrus aurantifolia (lime) oil, Melaleuca alternifolia (tea tree) leaf oil, Citrus grandis (grapefruit) peel oil, Citrus medica limonum (lemon) peel oil, rose flower oil, Eucalyptus globulus leaf oil, and combinations thereof.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 40 squalane;
(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is y 10, for instance Purcellin oil (cetearyl octanoate), isopropyl myristate, isopropyl palmitate, C12-C15 alkyl benzoate, such as the product sold under the trade name Finsolv TN™ or Witconol TN™ by Witco or Tegosoft TN™ by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI™ by Enichem Augusta Industriale, and also di(linear C14-C15 alkyl) tartrates, such as those sold under the name Cosmacol ETL™ by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid or linolenic acid;
(vii) carbonates, such as dicaprylyl carbonate, such as the product sold under the name Cetiol CC™ by Cognis;
(viii) fatty amides, such as isopropyl N-lauroyl sarcosinate, such as the product sold under the trade name Eldew SL 205™ from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

In some embodiments the cosmetic cleansing composition may include at least one branched or linear liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some embodiments, suitable liquid alkanes that may be used according to the disclosure include hydrocarbon-based oils containing from 8 to 16 carbon atoms, and especially branched C8-C16 alkanes such as C8-C16 isoalkanes. In some exemplary embodiments, such liquid alkanes may be chosen from isoparaffins, for instance isododecane (also known as 2,2,4,4,6-pentamethylheptane), isodecane, and isohexadecane.

In some embodiments, the cleansing composition are free or essentially free of oils and silicones. For example, the cleansing composition may in some embodiments include less than about 3%, less than about 2%, less than about 1%, or less than about 0.5% of one or more of oils, silicones or combinations thereof. In other embodiments, the cleansing composition comprise one or more of oils, silicones or combinations thereof, for example in an amount of from about 0.1% up to about 1%, such as up to about 2%, up to about 3%, up to about 4%, or up to about 5%. Non-limiting examples of silicones include amine-functionalized silicones (e.g., amodimethicone), dimethicone, bis-aminopropyl dimethicone, trimethyl silylamodimethicone, etc.

In some embodiments, the cleansing composition is free or essentially free of one or more of oils, silicones or combinations thereof.

In accordance with some embodiments, the cleansing composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), polyols, glycols, and combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and combinations thereof.

In accordance with the disclosure, one or more humectants may be present in the cleansing composition. In some embodiments, the humectant may comprise one or more of polyols, including, for example, glycerin, glycerol, glycols, such as, caprylyl glycol, butylene glycol, propylene glycol, isoprene glycol, dipropylene glycol, hexylene glycol and polyethylene glycols, monoethylene glycol, diethylene glycol, triethylene glycol, diethylene glycol, hexylene glycol, glycol ethers, such as, monopropylene, dipropylene and tripropylene glycol alkyl(C1-C4)ethers, squalane, triacetin, sugars, such as, glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, and seaweed extract.

In accordance with the various embodiments, the amount of humectant or conditioning agent present in the cleansing composition can range from about 1% to about 10%, or from about 1% to about 8%, or from about 1% to about 5%, or from about 2% to about 3%, or any suitable combination, sub-combination, range, or sub-range thereof by weight, based on the weight of the cleansing composition. One of ordinary skill in the art, however, will appreciate that other ranges are within the scope of the invention. One of ordinary skill in the art, however, will appreciate that other ranges are within the scope of the invention.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, in some embodiments that include optional additives, such optional additives will not materially adversely affect the cleansing composition.

### EXAMPLES

The following examples are intended to further illustrate the present disclosure. They are not intended to limit the disclosure in any way. Unless otherwise indicated, all parts are by weight.

### Example 1: Raw Materials

An embodiment of the inventive cleansing composition is provided in Table 1.

The examples below according to the invention are given by way of illustration and without a limiting nature. The names are the chemical name or the INCI name. Compositions and systems as described in the representative embodiments herein are selected from commercially available materials, including, as shown in the exemplified embodiments herein below.

Embodiments of the inventive cleansing composition as exemplified herein include the foregoing raw materials wherein the active constituents of the raw materials are listed as discrete ingredients and the amounts of active ingredients are given for the exemplified compositions as % by weight of the active ingredients based on the weight of the entire composition, and not as amounts of raw materials except as may be expressly stated

TABLE 1

| Raw Materials | |
|---|---|
| | PERCENT ACTIVE |
| RM INGREDIENT | (IF <100%) |
| COCAMIDOPROPYL HYDROXYSULTAINE | ~50% active |
| SODIUM METHYL COCOYL TAURATE | ~25% active |
| POLYQUATERNIUM-47 | ~20% active |
| TETRASODIUM GLUTAMATE DIACETATE | ~5% active |

### Example 2: Compositions

Exemplary embodiments of the cleansing composition according to the disclosure are set forth in Table 2, Comparative compositions are set forth in Table 3, and a comparative Benchmark composition is set forth in Table 3, all below.

TABLE 2

| | Inventive and Comparative Compositions | | | |
|---|---|---|---|---|
| | Inventive 1 | | Inventive 2 | Inventive 3 |
| Ingredient | w/2% SA | | w/1% SA | w/1.5% SA |
| SALICYLIC ACID; CAPRYLOYL SALICYLIC ACID | ~2.05 | % | 1.05 | 1.55 |
| LAURYL LACTATE | ~0.5-0.9 | % | | |
| COCAMIDOPROPYL HYDROXYSULTAINE* | ~3-10 | % | | |
| SODIUM METHYL COCOYL TAURATE * | ~3-10 | % | | |
| POLYQUATERNIUM-47 | ~0.05-0.5 | % | | |
| GLYCERIN; HEXYLENE GLYCOL | ~3-20 | % | | |
| SODIUM HYDROXIDE; MENTHOL; ZINC | ~2-20 | % | | |

| | | | |
|---|---|---|---|
| GLUCONATE; TETRASODIUM GLUTAMATE | | | |
| DIACETATE **; SODIUM BENZOATE; | | | |
| SODIUM CHLORIDE | | | |
| WATER | QS (~80) | QS (~80) | QS (~80) |

### Inventive 1

The inventive 1 composition includes salicylic acid (2); capryloyl salicylic acid (0.5), lauryl lactate (0.65), cocamidopropyl hydroxysultaine (7.5), sodium methyl cocoyl taurate (4.8), polyquaternium-47 (0.105), glycerin (5); hexylene glycol (0.5), sodium hydroxide; menthol; zinc gluconate; tetrasodium glutamate diacetate; sodium benzoate; sodium chloride (total of ˜3-˜0.4, 0.1, 0.2, 0.05, 0.001, 2.1, respectively), all amounts percentage (%) by weight, based on the weight of the composition.

TABLE 3
Comparative Compositions

| Comparative | Ingredients | Difference |
|---|---|---|
| COMP 1 | SAME AS | LACKS lauryl lactate |
| | INVENTIVE 1 | |
| COMP 2 | SAME AS | SUBSTITUTE sodium methyl |
| | INVENTIVE 1 | cocoyl taurate and cocamidopropyl |
| | | hydroxysultaine |
| | | with coco betain |
| COMP 3 | SAME AS | SUBSTITUTE polyquaternium |
| | INVENTIVE 1 | with chitosan |
| COMP 4 | SAME AS | SUBSTITUTE polyquaternium |
| | INVENTIVE 1 | with PEI |
| COMP 5 | SAME AS | SUBSTITUTE polyquaternium |
| | INVENTIVE 1 | with polylysine |

TABLE 4
Benchmark composition (PEG, and sulfate surfactant containing
cleanser that lacks cleansing composition of the invention)

| | Concentration |
|---|---|
| Ingredients | (% Active) |
| Actives | 2.05 |
| (Salicylic Acid, Capryloyl Salicylic Acid) | |
| Amphoteric Surfactants | 2.1 |
| (Coco Betaine) | |
| Nonionic surfactants | 4.3 |
| (Decyl Glucoside, Peg-150 Pentaerythrityl | |
| Tetrastearate (and) Peg-6 Caprylic/Capric | |
| Glycerides) | |
| Anionic surfactants | 10.14 |
| (Sodium Laureth Sulfate) | |
| Conditioning agents - Moisturizer | 3 |
| (Glycerin) | |
| Preservatives | 0.37 |
| (Tetrasodium EDTA, Zinc Gluconate) | |
| pH adjuster, Salts | ~2.5 |
| (Sodium chloride, Sodium Hydroxide, | |
| Citric acid) | |
| Viscosity Adjuster, Polymer | 3.1 |
| (Hexylene Glycol, POLYQUATERNIUM-47) | |
| Cooling Agent | 0.1 |
| (Menthol) | |
| Water | q.s. to 80 |

### Example 3: Stability

Each composition was evaluated for stability, including visual indicia of stability including precipitation, hazing; viscosity; and deposition on skin like substrate.

Each of the listed inventive and comparative formulations were evaluated visually by the unaided eye for clarity (haze) and stability at two months in ambient 25° C. conditions, wherein the comparatives each were varied from Inventive 1 in that they lacked lauryl lactate, or one or more ingredients were substituted, as indicated.

TABLE 5
Stability Results

| | Performance (Formulations evaluated |
|---|---|
| Composition | after 2 months at 25° C.) |
| INV 1 | No Phase Separation; 100% transparency; |
| | No re-crystallization of SA |
| INV 2 | No Phase Separation; 100% transparency; |
| | No re-crystallization of SA |
| INV 3 | No Phase Separation; 100% transparency; |
| | No re-crystallization of SA |
| Benchmark | No Phase Separation; 100% transparency; |
| | No re-crystallization of SA |
| COMP 1 | Unstable: Phase Separation; |
| | Hazy; re-crystallization of SA |
| COMP 2 | Unstable: Phase Separation; |
| | Poor SA deposition on skin |
| COMP 3 | Unstable: Phase Separation |
| COMP 4 | Unstable: Phase Separation |
| COMP 5 | Unstable: Phase Separation |

Referring to the results shown in Table 5, the comparatives all demonstrated instability at 2 months, while the tested embodiments of the inventive composition remained stable beyond two months, and further demonstrated stability at 45° C. after a period of three months. In addition, only the inventive formulations demonstrated phase stability, and clarity (lack of haze-being posited to represent maintained solubilization of the salicylic acid om the presence lauryl lactate), and satisfactory deposition of SA on skin. the latter.

Results with the substituted cationic agents surprisingly and unexpectedly show that mere inclusion of any cationic agent, such as a nature based cationic agent such as chitosan or a polyamine, is not sufficient to provide a stable composition. Likewise, results with the substituted surfactants surprisingly and unexpectedly show that an amphoteric surfactant such as coco betaine alone is insufficient to provide a stable composition, while a combination of amphoteric and anionic surfactants provide a stable composition.

Embodiments of the inventive compositions were also evaluated vs the benchmark composition for comparative performance of stability and deposition of salicylic acid on skin. Both the inventive and benchmark compositions demonstrated stability. After rinsing using Bio skin in vitro substrates, positive retention of salicylic acid on skin was observed for both inventive and benchmark with comparable results. These results vs benchmark demonstrate unexpectedly that a composition lacking sulfates and PEG based surfactants can stabilize high amounts of salicylic acid.

### Example 4: Viscosity and Sa Deposition

Viscosity was measured in the UD units by viscometer Rheomat R180 using an M3 spindle for ˜10 minutes at 25° C. Stable inventive compositions demonstrate viscosity in a range from about 15 UD to about 50 UD (M3). In some particular embodiments, the viscosity is in the range from about 25 UD to about 35 UD. Unsatisfactory compositions are deemed unstable at viscosities that fall below 15 UD or exceed 50 UD. For example, as described herein, use of certain surfactants, such as coco-betaine alone in the absence of other surfactants, resulted in unstable and/or insufficient viscosity.

### Salicylic Acid Deposition Test

Processing of BIOSKIN Samples: BIOSKIN samples of about 2 in by 2 inches were provides. Each BIOSKIN sample was treated with one of an inventive, example and comparative composition by application of about 0.5 g of the tested composition with a cleansing brush (Clarisonic™ brush). Cleansed samples were soaked in water for about 30 seconds in 40° C. water, then massaged using the cleansing brush for about one minute. The samples were then rinsed under water at in 40° C. water then air dried for about an hour. Samples were then soaked in a solution of iron chloride (0.01% FeCl₃) for about an hour then air dried overnight.

The color of each sample of treated BIOSKIN (vs a control sample lacking any treatment) was measured using a hand-held colorimeter and the measurements were analyzed to provide a color change (resulting from binding to the iron compound to deposited SA) reflected as a percentage change in color relative to the control. The results are shown in Table 6, below.

TABLE 6
Viscosity And Salicylic Acid Deposition with Inventive, Comparative and
Benchmark Compositions

| | Benchmark | Inventive 1 | | | Comparative 1 |
|---|---|---|---|---|---|
| | Sulfate | Lauryl | Inventive 2 | Inventive 3 | W/O Lauryl |
| | Surfactant + | Lactate + 2% | Lauryl Lactate + | Lauryl Lactate + | Lactate + 2% |
| | 2% SA | SA | 1% SA | 1.5% SA | SA |
| % Color | 16.89 | 19.46 | 17.33 | 18.25 | 11.35 |
| Change | | | | | |
| Average | | | | | |
| Viscosity | 15.6 | 35.7 | 30.5 | 33.1 | <0 out of |
| (M3) UD | | | | | range |

Referring to the results shown in Table 5, it is clear that each embodiment of the inventive composition demonstrates a significant level of deposition of SA on the synthetic skin, based on the measured color change, and the extent of deposition is comparable to the benchmark. Further each embodiment of the inventive composition demonstrates viscosity in a range from about 15 UD to about 50 UD which is required to confer retention on skin and resistance to runniness prior to rinsing, while the comparative composition is far more liquidous, demonstrating a negligible viscosity, and thus unsuitable for retention on skin prior to rinsing, and far less effective for deposition of SA on skin. Further, while the Benchmark demonstrates viscosity in an acceptable range, it is less than half of the viscosity of the embodiments of the inventive compositions, all of which have a viscosity of greater than 30 UD.

Accordingly, as shown in the above Examples, the inventive composition provides a stable, and sufficiently thick and spreadable architecture for ease of application to skin and associated retention of effective amounts of SA on skin after rinsing. This is surprisingly accomplished in the absence of PEG and sulfate containing ingredients that are commonly employed and accepted and understood in the art to be necessary for solubilizing and stabilizing large amounts of SA, which ingredients are expressly excluded from the inventive composition.

While the disclosure has been described with reference to described embodiments, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

“Active material” as used herein with respect to the percent amount of an ingredient or raw material, refers to 100% activity of the ingredient in the raw material except as specifically stated. All amounts given herein are relative to the amount of active material, unless otherwise indicated.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one” or “one or more” as used herein, means that there may be one, two, three or more and thus includes individual components as well as mixtures/combinations.

The transitional terms “comprising,” “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step, or material. The term “consisting of” excludes any element, step, or material other than those specified in the claim, and, in the latter instance, impurities ordinarily associated with the specified material(s). The term “consisting essentially of” limits the scope of a claim to the specified elements, steps, or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., may be modified by the term “about.” Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated. As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc. As used herein a range of ratios is meant to include every specific ratio within, and combination of subranges between the given ranges.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A cleansing composition comprising:
a) at least one acne treatment active comprising at least one beta hydroxy acid that comprises salicylic acid or a derivative thereof, present in an amount that is greater than 1%, by weight, based on the weight of the cleansing composition; and
b) an aqueous carrier system comprising:
(i) at least one thickener comprising a lauryl lactate;
(ii) at least one surfactant;
(iii) at least one cationic agent; and
(iv) water,
wherein the cleansing composition is essentially free of PEG-type compounds, PEG-type surfactants, and sulfate-based surfactants.

2. The cleansing composition according to claim 1, wherein salicylic acid is present in an amount that is in a range from greater than 1% to about 4%, by weight, based on the weight of the cleansing composition.

3. The cleansing composition according to claim 1, wherein salicylic acid is present in an amount that is in a range from greater than 1% to about 2%, by weight, based on the weight of the cleansing composition.

4. The cleansing composition according to claim 1, wherein lauryl lactate is present in the cleansing composition from about 0.2% to about 5%, by weight based on the weight of the composition

5. The cleansing composition according to claim 1, wherein lauryl lactate is present in the cleansing composition at about 0.65%, by weight based on the weight of the composition.

6. The cleansing composition according to claim 1, wherein the at least one surfactant comprises at least one amphoteric surfactant and at least one anionic surfactant each present in an amount that is in a range from about 0.05% to about 15%, by weight, based on the weight of the cleansing composition.

7. The cleansing composition according to claim 6, wherein the at least one amphoteric surfactant includes cocamidopropyl hydroxysultaine present in the cleansing composition from about 1% to about 10%, and the at least one anionic surfactant includes sodium methyl cocoyl taurate present in the cleansing composition from about 1% to about 10%, by weight based on the weight of the composition.

8. The cleansing composition according to claim 6, wherein the at least one amphoteric surfactant includes cocamidopropyl hydroxysultaine present in the cleansing composition at about 7.5%, and the at least one anionic surfactant includes sodium methyl cocoyl taurate present in the cleansing composition at about 4.8%, by weight based on the weight of the composition.

9. The cleansing composition according to claim 1, wherein the at least one cationic agent is polyquaternium-47 present from about 0.01% to about 5%, by weight based on the weight of the composition.

10. The cleansing composition according to claim 1, wherein the at least one cationic agent is polyquaternium-47 present at about 0.1%, by weight based on the weight of the composition.

11. The cleansing composition according to claim 1, wherein the cleansing composition, when applied to a keratinous tissue, demonstrates deposition and retention of the beta hydroxy acid on the keratinous tissue after rinsing with water.

12. The cleansing composition according to claim 1, wherein the cleansing composition exhibits stability from phase separation and crystallization of the beta hydroxy acid at an ambient temperature of about 45° C. for a period of at least 3 months.

13. The cleansing composition according to claim 1, wherein the cleansing composition is free of Decyl Glucoside, Peg-150 pentaerythrityl tetrastearate (and) Peg-6 caprylic/capric glycerides, sodium lauryl sulfate, sodium laureth sulfate, sodium lauryl ether sulfate, ammonium lauryl sulfate, ammonium laureth sulfate.

14. The cleansing composition according to claim 1, further comprising one or more additives selected from the group consisting of skin care actives, humectants, water-based solvents, pH adjusters, viscosity adjusters, chelating agents, preservatives, cooling agents, fillers, fragrances, dyes, pigments, oils, and combinations thereof.

15. The cleansing composition according to claim 1, further comprising one more additives selected from the group consisting of capryloyl salicylic acid, glycerin, sodium hydroxide, zinc gluconate, tetrasodium glutamate diacetate, sodium benzoate, hexylene glycol, sodium chloride, and menthol, and combinations thereof.

16. A cleansing composition comprising:
a) at least one acne treatment active comprising at least one beta hydroxy acid comprising salicylic acid present in an amount that is greater than 1%, by weight, based on the weight of the cleansing composition; and
b) an aqueous carrier system comprising:
(i) at least one thickener comprising a lauryl lactate present in an amount that is in a range from about 0.1% to about 1%;
(ii) a combination of surfactants comprising at least one amphoteric surfactant and at least one anionic surfactant each present in an amount that is in a range from about 0.05% to about 15%, by weight, based on the weight of the cleansing composition;
(iii) at least one cationic agent present in an amount that is in a range from about 0.01% to about 0.5%, by weight, based on the weight of the cleansing composition; and
(iv) water,
wherein the cleansing composition has a viscosity is in a range from about 15 UD to about 50 UD, and is essentially free of PEG-type compounds, PEG-type surfactants, and sulfate-based surfactants.

17. The cleansing composition according to claim 16, wherein the cleansing composition, when applied to a keratinous tissue, demonstrates deposition and retention of the beta hydroxy acid on the keratinous tissue after rinsing with water.

18. The cleansing composition according to claim 16, wherein the cleansing composition exhibits stability from phase separation and crystallization of the beta hydroxy acid at an ambient temperature of about 45° C. for a period of at least 3 months.

19. The cleansing composition according to claim 16, wherein the cleansing composition is free of Decyl Glucoside, Peg-150 pentaerythrityl tetrastearate (and) Peg-6 caprylic/capric glycerides, sodium lauryl sulfate, sodium laureth sulfate, sodium lauryl ether sulfate, ammonium lauryl sulfate, ammonium laureth sulfate.

20. A cleansing composition comprising:
a) at least one acne treatment active comprising at least one beta hydroxy acid comprising salicylic acid present in an amount that is from about 1% to about 2%, by weight, based on the weight of the cleansing composition; and
b) an aqueous carrier system comprising:
(i) at least one thickener comprising a lauryl lactate present at about 0.65%;
(ii) a combination of surfactants comprising at least one amphoteric surfactant that includes cocamidopropyl hydroxysultaine present in the cleansing composition at about 7.5%, and at least one anionic surfactant that includes sodium methyl cocoyl taurate present in the cleansing composition at about 4.8%, by weight, based on the weight of the cleansing composition;
(iii) at least one cationic agent that includes polyquaternium-47 present at about 0.1%, by weight based on the weight of the composition;
(iv) additives comprising capryloyl salicylic acid present at about 0.05%, glycerin present at about 5%, sodium hydroxide present at about 0.4%, zinc gluconate present at about 0.2%, tetrasodium glutamate diacetate present at about 0.05%, sodium benzoate present at about 0.001%, hexylene glycol present at about 0.5%, sodium chloride present at about 2.1%, and menthol present at about 0.1%, all by weight based on the weight of the composition; and
(v) water;
wherein the cleansing composition has a viscosity is in a range from about 25 UD to about 35 UD, and is essentially free of PEG-type compounds, PEG-type surfactants, and sulfate-based surfactants.
